(19) United States
(12) Patent Application Publication
(10) Pub. No.: US 2015/0128962 A1
(43) Pub. Date: May 14, 2015

(54) MAGNETIC SYSTEM FOR ATTACHING AND REMOVING MEDICAL DEVICES AND PROSTHESES ON HUMAN SKIN

(71) Applicant: CENTRO BRASILEIRO DE PESQUISAS FISICAS - CBPF, Rio de Janeiro (BR)

(72) Inventors: Joao Paulo Sinnecker, Rio de Janero (BR); Michel Luciano Holder Toledano Vaena, Rio de Janeiro (BR)

(21) Appl. No.: 14/401,576

(22) PCT Filed: May 16, 2013
(86) PCT No.: PCT/BR2013/000169

§ 371 Date: Nov. 17, 2014

(30) Foreign Application Priority Data
May 18, 2012 (BR) BR1020120118998

(51) Int. Cl. A61F 2/78; A61F 5/449

(52) U.S. Cl. CPC A61F 2/78; A61F 5/449; A61F 2002/7868
USPC 128/899

(57) ABSTRACT

The present invention relates to a magnetic system for attaching and removing medical devices and prostheses on human skin comprising external disks 4 and silicone implants 5; said system can increase, decrease or eliminate the magnetic attraction or repulsion force as a function of the rotation of the external disks 4 in relation to the silicone implants 5 such that the skin between the external disks 4 and the silicone implants 5 is subject to a pressure compatible with viable capillary microcirculation.

### 1) TECHNICAL FIELD

This application belongs to the technical field of apparatuses that are exclusively for medical use, for attaching medical devices and prostheses on human skin.

### 2) PRIOR ART

The human skin is composed of subcutaneous tissue (1) or hypodermis tissue (the innermost layer), the dermis (2) which is the intermediate layer and the epidermis (3), the topmost layer. The hypodermis (1) and the dermis (2) provide certain physical skin properties (such as elasticity and flexibility), while the epithelium of the epidermis (3) is responsible for certain physiological functions of the skin (such as a barrier against microbial invasion or prevention of water loss).

An ostomy (or stoma) is a surgical opening in the skin so that physiological eliminations may be expelled from the body. An ostomy allows these eliminations to leave the intestines (colostomy or ileostomy) or bladder (urostomy). The ostomy may be permanent or transient, and in the latter case, the patient awaits a definitive surgical reconstruction. As most stoma eliminate noxious substances, the skin around the stoma must be protected from its harmful effects. A bag or container is attached to the stoma in order to retain these noxious products such as digestive enzymes, urine or feces.

Current methods to care for ostomies involve the use of bags that are attached to the skin around the stoma using adhesives. The repeated application and removal of these adhesives can damage the skin around the stoma or even lead to allergic reactions. The current pouches (ostomy bags) may consist of one or two pieces. In the single-piece pouches, there is an adhesive surface on one side of the collection bag itself. In the pouches made of two parts, there is an adhesive sheet, in contact with skin, into which the collection bag fits, making it possible to change the bag without needing to remove the adhesive sheet, thus sparing the skin. However, these adhesive sheets must periodically be removed since urine or feces can infiltrate underneath them.

A method of securing the ostomy collection bags through the use of magnetic force has been described by Giesy (U.S. Pat. No. 3,565,073) in which a magnetic ring is implanted through a surgical incision in the skin around the stoma. Adair (U.S. Pat. No. 4,205,678) describes the introduction of several magnetic implants underneath the skin around the stoma through pinpoint incisions using a trocar, which eliminates the need for large open incisions.

In addition to the stoma, the skin is subject to wounds, which can occur for several reasons: trauma, surgery, infection, etc. The majority of wounds to the skin heal within a few days or weeks. Some wounds, however, may remain open for long periods of time, such as with ionizing radiation wounds, vascular leg ulcers or pressure sores. Such chronic wounds require daily dressing changes over months or years. The dressings may contain elements to protect against infections (such as antibiotics or silver ions) or elements to maintain a moist environment within the non-epithelial wound (such as hydrocolloids or hydrogels). These special conditions promote healing and improve and accelerate the healing process.

During the healing phase, attaching local dressings to accelerate wound healing is usually done with tape or bandages. Since most dressings require daily change, the repeated application and removal of adhesive tapes can damage the skin adjacent to the wound, previously unscathed. Also, bandages that are too slackly applied tend to loosen and fall off with the movement of the body, and tight bandages may lead to local circulatory restrictions. When the wound is finally closed and re-epithelized, the dressings are no longer needed and the healing process is finished, resulting in a scar.

In some individuals, however, a pathological healing process occurs in which the scar tissue grows larger than usual, resulting in an aesthetically undesirable scar. When this abnormal scar remains within the boundaries of the original wound, is called a hypertrophic scar. When it grows beyond the original wound, it is called keloid. Although keloids are not considered to be tumors in the strictest sense, in practice, keloids present a tumor-like behavior, since their growth is difficult to control. In addition to the unsightly appearance, keloids and hypertrophic scars may cause other unwanted symptoms (such as pain, itching, burning sensation), causing great suffering for the affected patients.

There are several treatments recommended for keloids and hypertrophic scars. These treatments may be pharmacological, surgical or physical in nature. After surgical removal of the keloid, the new scar is treated by physical, non-surgical adjuvants, to reduce the chance of recurrence. These adjunctive treatments may include the application of early postoperative radiation therapy, the use of pressure therapy (applying mechanical pressure on the scar through elastic garments, belts or compressive dressings) and the use of silicone sheets on the scar.

After the initial phase of healing, treatment with pressure (pressure therapy) is carried out with the use of bandages, elastic mesh or compression garments, which can be very inconvenient because they are tight, hot, and cause sweating of the patient as well as the ungainly appearance of these garments. The silicone sheets are applied directly on the scar, but attaching them to the desired anatomical region is problematic because perspiration and skin oils tend to make the sheets move from its original location. These silicone sheets may be secured with tape or bandages, but this causes the same drawbacks as mentioned above.

A method of securing the silicone sheets through the use of magnetic force was described by Vaena (WO 2012/006702). The author describes the use of magnets within the silicon sheet, which is held on the skin due to the magnetic attraction to implants surgically placed under the skin. In this case, the magnetic force that attracts the silicon sheet also generates a constant and uniform pressure on the scar, bringing additional benefits in the treatment of keloids and hypertrophic scars, since the pressure therapy combines with the continuous contact of the silicon sheets. Another author, Li Yiguo (CN2707294Y) describes the use of a single magnet in wound healing, defending the alleged properties of magnetism as a promoter of healing, which would speed healing and reduce pain, there is however no supporting data in the scientific literature. In other words, Li Yiguo does not use the magnetic properties of a magnet to generate mechanical pressure on the scar.

The use of magnetic force for attaching prostheses is widespread. Attaching dental prostheses with magnetic devices has been described by Shiner et al. (U.S. Pat. No. 4,997,372). Many mutilated patients who have experienced the loss of the nose (or ear), for example, make use of a magnetic nasal prosthesis (or ear), which in turn is attached to magnetic osseointegrated implants (attached to the bone) through titanium pins. Eaton (PI0008270-8 A) describes attaching an external breast prosthesis to the skin of the user via magnets (contained in the prosthesis) which would be attracted to steel buttons surgically implanted under the skin of the patient.

The surgical incisions located very close to the place of residence of the implant increases the chance of complications such as infection, migration or extrusion of the implant through the scar. To minimize the chance of implant migration through the scar, Eaton (PI0008270-8 A) suggests the use of a biocompatible Teflon fabric (or similar) under the scar so as to strengthen the skin over the implant. The extrusion problem can be circumvented through use of pinpoint incisions far away from the implant, inserting the implants with the use of a trocar, as proposed by Adair (U.S. Pat. No. 4,205,678) and Vaena (WO 2012/006702).

The integrity of the skin located between the inner magnetic pole (subcutaneous implant) and the external pole (above the skin) can be compromised, since this skin is subjected to continuous pressure, leading to pain in the cutaneous microcirculation and can lead to skin atrophy and even necrosis. To avoid such complications, Vaena (WO 2012/006702) discloses a method for periodic relief of this pressure by periodic rotation of the external device, since the arrangement of the magnets is performed alternately, with intermittency at the pressure points, to allow circulatory reestablishment of the skin.

### 3) PROBLEM FOUND IN THE PRIOR ART

All current methods using magnetic force to attach prostheses or medical devices on human skin have some limitations:
a) Upon withdrawal of the medical device (or prosthesis) from its attachment location, the magnetic force tends to attract the implants that are under the skin, forcing the implants to exit outside the human body, which can cause pain and discomfort to the user.
b) Each time the medical device (or prosthesis) is removed from its position, the implants force their way out through the skin. If the scar is closed, there may be an extrusion of the implant through the scar. If the scar is remote, the force exerted repeatedly by the implants (from the inside out) in the skin will cause a progressive thinning thereof, with a risk of atrophy and necrosis.
c) The periodic relief of pressure on the skin, by periodically rotating the external device, as proposed by Vaena (WO 2012/006702), may mitigate this tendency to atrophy, but will not solve it, since each periodic rotation of the external device necessarily incurs the removal of the device from its original position, forcing the exit movement of the implant through the skin, causing the same disorders as mentioned above.

### 4) OBJECTIVE OF THE INVENTION

The objective of the invention is to solve the limitations previously mentioned, making use of a system that allows the reduction, cancel out or reverse the direction of the magnetic force of attraction, to allow the attachment and removal of medical devices (such as ostomy collection bags, dressings and silicone sheets) and prostheses on the outer surface of human skin in a way that is not traumatic.

### 5) DESCRIPTION OF THE FIGURES

FIG. 1 is a representative diagram in perspective of the silicone implant (5) with magnets (6) inside.

FIG. 2 is a representative diagram of the human skin with its epidermal constituents (3), the dermis (2) and the hypodermis (1), with the external disk (4), with its magnets (6) having axial magnetization, on top of the skin and the silicone implant (5) with their respective axially magnetized magnets (6) under the skin.

FIG. 3 is a representative diagram of the human skin with its epidermal constituents (3), the dermis (2) and the hypodermis (1), with the external disk (4), with its respective external diametrically magnetized magnet (7), on top of the skin and the silicone implant (5) with their respective diametrically magnetized internal magnets (8) under the skin.

FIG. 4 is a representative diagram of the human skin with its epidermal constituents (3), the dermis (2) and the hypodermis (1), upon which is a plate for fastening triangularly shaped ostomy collection bags (9), with three external disks (4) containing their respective axially magnetized magnets (6) and under which there are three silicone implants (5) containing its axially magnetized magnets (6).

FIG. 5 is a representative diagram of a capillary prosthesis (10) with an external disk (4) and silicone implant (5) under the skin.

FIG. 6 is a representative diagram of a scar (11) in human skin with its epidermal constituents (3), the dermis (2) and the hypodermis (1), which is on one rectangularly shaped silicon plate (12), with three external disks (4) containing its respective axially magnetized magnets (6) and under which there are two silicone implants (5) containing its axially magnetized magnets (6).

### 6) DESCRIPTION OF THE INVENTION

The invention in question is a system formed by one or more external disks (4) and silicone implants (5), each containing a variable number of magnets (6), which may be axially or diametrically magnetized, for attaching and removal of medical devices and prostheses to human skin.

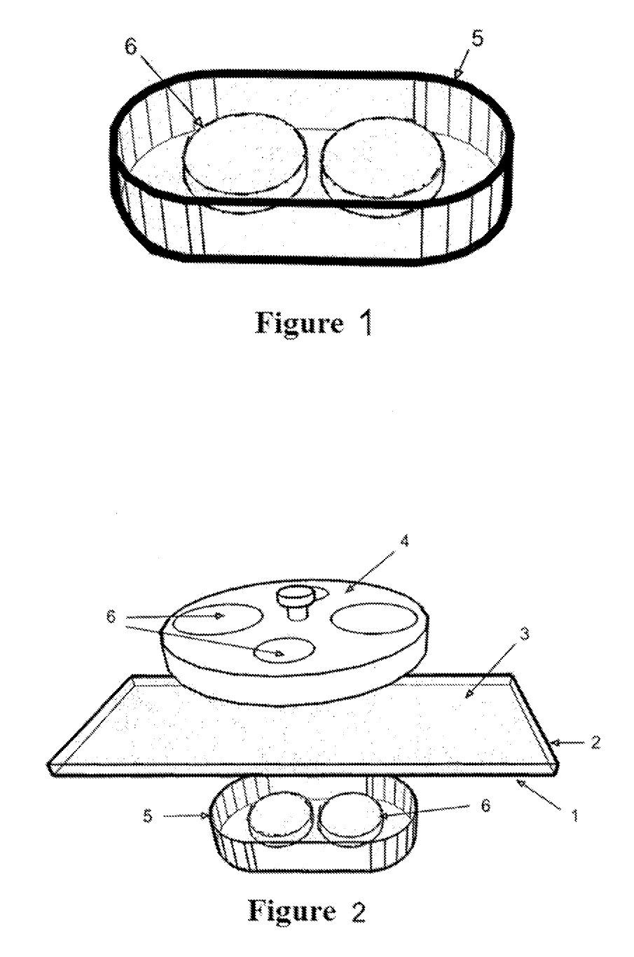

Said silicone implant (5) contains magnets (6) in a linear array. The magnets (6) are wrapped in biocompatible silicone, resulting in a narrow, elongated, flattened shaped implant (5), with rounded edges. In order to minimize tissue trauma and reduce the risk of extrusion, the implant should be flexible and have a smooth surface, with no corners or edges, as illustrated in FIG. 1.

This silicone implant (5) may be inserted under the skin with the aid of a trocar, as described by Vaena (WO 2012/006702), through a minimal skin incision and blunt dissection at the subdermal level, by configuring a percutaneous route. Alternatively, this implant may also be inserted through open surgery.

The medical device (or prosthesis) that is to be attached to and removed from human skin contains one or more outer disks (4), containing a variable number of magnets (6). These magnets (6) may be axially or diametrically magnetized in accordance with the type of magnet (6) used in the implant (5).

In the case of axial magnetization (6), the magnets are arranged in a radial configuration relative to the central axis of the disk. The rotation of the outer disk (4) allows an attractive or repulsive configuration between the magnetic pole of the magnet (6) in the outer disk (4) and the magnetic pole of the magnet (6) contained in the silicone implant (5). When we want to fix the medical device or prosthesis to the skin, we will use the attractive configuration in which there is an attraction between opposite poles. When detachment is desired, the disk is rotated to the repulsive configuration in which there is equal repulsion between two magnetic poles, as shown in FIG. 2.

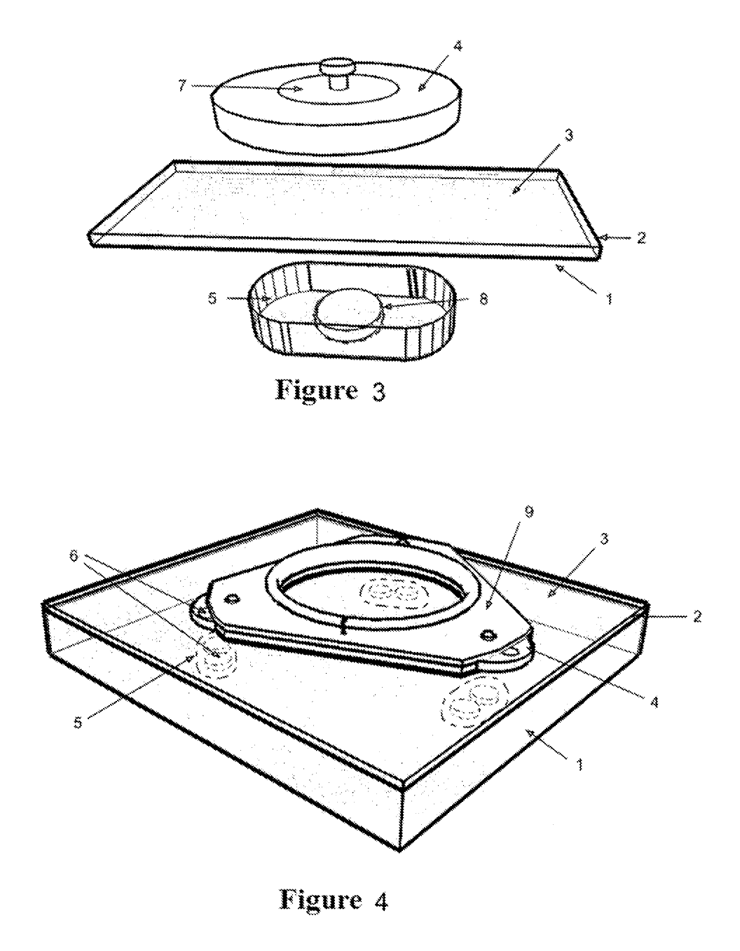

In the case of diametrical magnetization, a single diametrically magnetized magnet occupies a central position in the external disk (4) and rotating it will allow an attractive or repulsive configuration between the external diametrically magnetized magnet (7) contained in the outer disk (4) and the inner diametrically magnetized magnet (8) contained in the silicone implant (8), as illustrated in FIG. 3.

The pressure on the dermal-epidermal layer interposed between the magnets (6) of the outer disk (4) and magnets (6) of the silicone implants (5) should not excessively exceed the normal capillary pressure so as to maintain blood flow in the subdermal plexus. As the dermo-epidermal layer interposed between the magnetic poles is subjected to sustained pressure, circulation may be compromised and there may be local skin changes, with progressive dermal atrophy. These alterations of the skin may lead to undesirable complications, such as skin necrosis or extrusion. To avoid such complications, periodic relief of this pressure is recommended, in order to allow reestablishment of circulation to the skin. Relief may be obtained by periodically rotating the external disk (4).

In the case of axial magnetization, we can employ magnets (6) having a greater or lesser magnetic moment in each segment of the external disk (4), thus calibrating the pressure on the skin.

If the absence of force is necessary, a certain segment of the external disk (4) may not contain its magnet (6), determining a neutral setting (not attractive and not repulsive) in relation to the magnets (6) contained in the silicone implant (5).

In the case of diametric magnetization, rotation of the disk will determine the strength of the magnetic attraction in each position, calibrating the pressure exerted on the skin. If the absence of force is required, the external disk (4) must remain in a neutral position (not attractive and not repulsive), with no attraction or repulsion between the external diametrically magnetized magnets (7) of the outer disk (4) and the internal diametrically magnetized magnet (8) contained in the silicone implant (5).

This system allows attaching and removing medical devices on human skin, such as an attachment plate for an ostomy collection bag (9), as illustrated in FIG. 4. Rotating the external disks (4) to a repulsive configuration allows for safe removal of this attachment plate for an ostomy collection bag (9) in a painless and non-traumatic way for the patient.

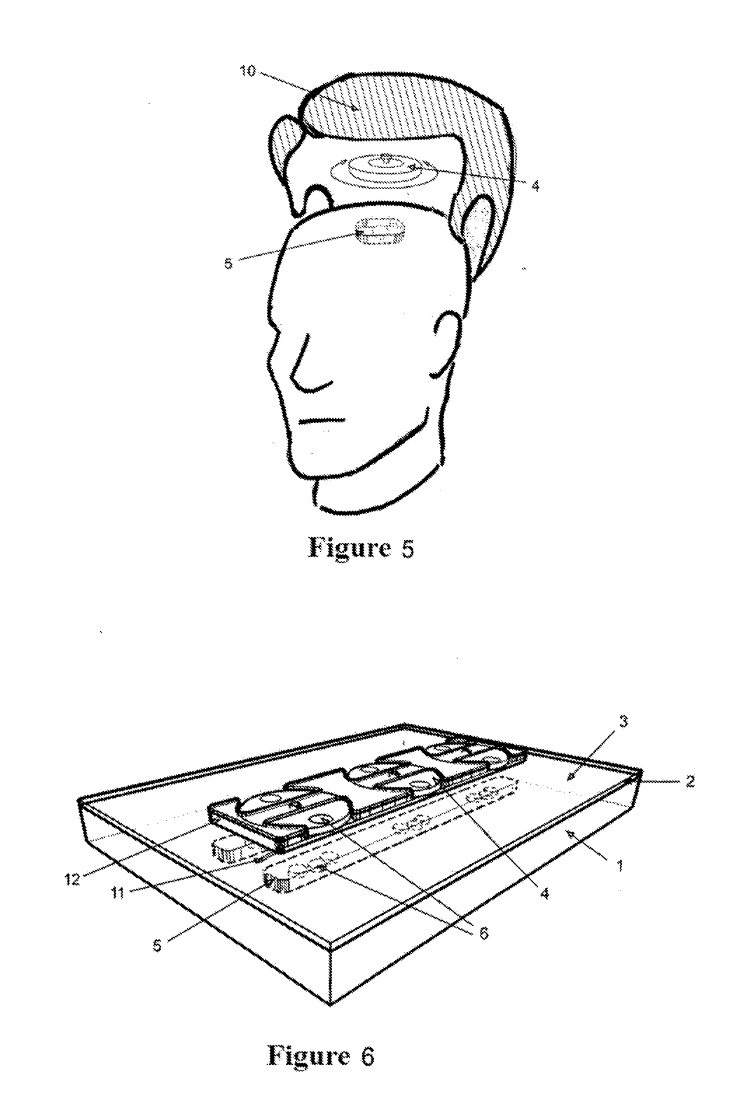

In the case of prostheses, the system may be employed to secure a capillary prosthesis (10)—or wig—as illustrated in FIG. 5. The rotation of the outer disk (4) to a repulsive configuration allows for safe removal of the hair prosthesis (10) when the user wants to take a shower, for example.

Rotation of the outer disk (4) for an attractive setting enables secure attachment of the hair prosthesis (10) in situations of much movement, as in sports.

For the treatment of keloids and hypertrophic scars, two subdermal level silicone implants (5) may be inserted parallel to the scar (11), as illustrated in FIG. 6. The arrangement of the silicone implants (5) and external disks (4) with their respective magnets (6) occurs so as to promote the placement of a silicon sheet (12) on the wound (11) we want to treat.

Rotation of the external disks (4) makes it possible to switch the application points of force on the skin, while retaining all of the silicone sheet (12) pressing on the scar (11) in a constant manner, generating a pressure between 24 and 30 mmHg.

### 7) TECHNICAL TERMS

Adjuvants—Adjuvants—That which helps, assists. In medicine, it is said of a drug or treatment that enhances the action of another.
Atrophy—Atrophy—Wasting, wear, deterioration.
Corticosteroids—corticoids—Name given to a group of hormones produced by the adrenal cortex, their derivatives and synthetic substitutes, especially used as anti-inflammatories. Glucocorticoids. Glucocorticoids. Decubitus ulcers
Extrusion—Extrusion—Spontaneous expulsion from inside the body. Expulsion.
Hemostasis—Haemostasis—Action to contain a bleed. Stopping blood.
Hypertrophic—Hypertrophic—Refers to that involving hypertrophy.
Hypertrophy—Hypertrophy—Abnormal development of an organ tissue.
Necrosis—Necrosis—Cell, tissue or organ death.
Pathological—Pathological—Related to a particular disease. Morbid.
Keloids—Keloid—Fibrous, elongated tumor of the skin, resulting most often from a hypertrophic scar.
Radiotherapy—Radiotherapy—Therapeutic Medicine based on the use of radioactive energy. Treatment using radiation applications.
Relapse—Return—Reappearance of a disease after a more or less long period of healing.
Trocar—Trocar—Cannula used for punctures.

1. System for attaching and removing medical devices and prostheses on human skin characterized in that:
a variable number of external disks (4) and a variable number of silicone implants (5),
increase, decrease or reverse the magnetic force of attraction or repulsion due to the rotation of said external disks (4) with respect to said silicone implants (5),
subjecting the skin interposed between said outer disks (4) and silicone implants (5) to a pressure compatible with the viability of capillary microcirculation,
alternation in the pressure points on the human skin as due to the rotation of said external disks (4) with respect to said silicone implants (5).

2. External disk (4) characterized in that:
it contains a set of magnets (6):
which are axially or diametrically magnetized,
with a variable magnetic intensity,
wherein the position of each magnet (6) in relation to said external disk (4) is fixed,
wherein the arrangement of the set of magnets (6) in relation to said external disk (4) may vary,
presenting variable form and dimensions depending on the characteristics of the medical device or prosthesis which is connected.

3. Silicone implant (5) characterized in that:
it is flexible, biocompatible and sterilizable,
it possesses, in a fixed internal position, a variable number of magnets (6) being axially or diametrically magnetized, the intensity of the magnetization of these may vary as well as their arrangement,
it is of a narrow, elongated and flattened shape, with rounded borders, having no corners, sharp borders or edges,
it is implantable under human skin, subdermally or subcutaneously, using a trocar or through open surgery.
